SEQUENCE LISTING (4 sequences)

SEQ ID NO: 1 - DNA, 1536 bp, Bacillus amyloliquefaciens
  CDS (1)..(1536)
atgaaacaacaaaaacggctttacgcccgattgctgacgctgttatttgcgctcatcttcttgctgcctcattctgcagcagcggcggtaaatggcacgctgatgcagtattttgaatggtatacgccgaacgacggccagcattggaaacgattgcagaatgatgcggaacatttatcggatatcggaatcactgccgtctggattcctcccgcatacaaaggattgagccaatccgataacggatacggaccttatgatttgtatgatttaggagaattccagcaaaaagggacggtcagaacgaaatacggcacaaaatcagagcttcaagatgcgatcggctcactgcattcccggaacgtccaagtatacggagatgtggttttgaatcataaggctggtgctgatgcaacagaagatgtaactgccgtcgaagtcaatccggccaatagaaatcaggaaacttcggaggaatatcaaatcaaagcgtggacggattttcgttttccgggccgtggaaacacgtacagtgattttaaatggcattggtatcatttcgacggagcggactgggatgaatcccggaagatcagccgcatctttaagtttcgtggggaaggaaaagcgtgggattgggaagtatcaagtgaaaacggcaactatgactatttaatgtatgctgatgttgactacgaccaccctgatgtcgtggcagagacaaaaaaatggggtatctggtatgcgaatgaactgtcattagacggcttccgtattgatgccgccaaacatattaaattttcatttctgcgtgattgggttcaggcggtcagacaggcgacgggaaaagaaatgtttacggttgcggagtattggcagaataatgccgggaaactcgaaaactacttgaataaaacaagctttaatcaatccgtgtttgatgttccgcttcatttcaatttacaggcggcttcctcacaaggaggcggatatgatatgaggcgtttgctggacggtaccgttgtgtccaggcatccggaaaaggcggttacatttgttgaaaatcatgacacacagccgggacagtcattggaatcgacagtccaaacttggtttaaaccgcttgcatacgcctttattttgacaagagaatccggttatcctcaggtgttctatggggatatgtacgggacaaaagggacatcgccaaaggaaattccctcactgaaagataatatagagccgattttaaaagcgcgtaaggagtacgcatacgggccccagcacgattatattgaccacccggatgtgatcggatggacgagggaaggtgacagctccgccgccaaatcaggtttggccgctttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa

SEQ ID NO: 2 - protein, 512 aa, Bacillus amyloliquefaciens
MKQQKRLYARLLTLLFALIFLLPHSAAAAVNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 3 - DNA, 1434 bp, Aspergillus oryzae
  CDS (1)..(1434)
gcaacgcctgcggactggcgatcgcaatccatttatttccttctcacggatcgatttgcaaggacggatgggtcgacgactgcgacttgtaatactgcggatcagaaatactgtggtggaacatggcagggcatcatcgacaagttggactatatccagggaatgggcttcacagccatctggatcacccccgttacagcccagctgccccagaccaccgcatatggagatgcctaccatggctactggcagcaggatatatactctctgaacgaaaactacggcactgcagatgacttgaaggcgctctcttcggcccttcatgagagggggatgtatcttatggtcgatgtggttgctaaccatatgggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccattgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcg

SEQ ID NO: 4 - protein, 478 aa, Aspergillus oryzae
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

(19) United States
(12) Patent Application Publication
(10) Pub. No.: US 2009/0226564 A1
(43) Pub. Date: Sep. 10, 2009

(54) BACTERIAL ALPHA-AMYLASE VARIANTS

(75) Inventors: Steffen Danielsen, Copenhagen (DK); Henrik Lundqvist, Malmo (SE)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/305,081

(22) PCT Filed: Jun. 29, 2007
(86) PCT No.: PCT/EP2007/056590

§ 371 Date: Dec. 16, 2008

(30) Foreign Application Priority Data
Jun. 30, 2006 (DK) PA 2006 00891

(51) Int. Cl. A21D 8/04; C12N 9/28; C07H 21/04; C12N 15/63; C12N 1/20; C12P 21/06

(52) U.S. Cl. 426/18; 435/202; 536/23.2; 435/320.1; 435/252.3; 435/69.1; 426/61

(57) ABSTRACT

The present invention relates variants of parent Bacillus amyloliquefaciens alpha-amylases, notably variants exhibiting altered pH-profile, which are advantageous with respect to applications of the variants in baking.

### SEQUENCE LISTING AND DEPOSITED MICROORGANISMS

### Sequence Listing

The present invention comprises a sequence listing.

### Deposit of Biological Material

None.

### FIELD OF THE INVENTION

The present invention relates variants of parent Bacillus amyloliquefaciens alpha-amylases, notably variants exhibiting altered pH-profile, which are advantageous with respect to applications of the variants in baking.

### BACKGROUND OF THE INVENTION

Alpha-Amylases (alpha-1,4-glucan-4-glucanohydrolases, EC 3.2.1.1) constitute a group of enzymes which catalyze hydrolysis of starch and other linear and branched 1,4-glucosidic oligo- and polysaccharides.

The prior art suggests that fungal and cereal alpha-amylase preparations can be used for improving loaf volume and that bacterial and cereal alpha-amylase have also a crumb softening effect. Effective action of the alpha-amylase requires that the enzyme is stable enough to partially degrade the starch fraction during the baking process in order to obtain inter alia an increased volume, but on the other hand the activity of the enzyme should decrease at a certain stage of the baking process in order to avoid over dosage causing a gummy crumb.

EP 0409299 B1 provides a modified bacterial alpha-amylase which exhibits reduced thermostability under baking conditions relative to the corresponding parent enzyme and having an amino acid sequence which differs in at least one amino acid from the parent alpha-amylase at the amino acid number 113, 114, 116, 123, 163, 164, 166, 238, 316, 322, 345, 349, 386, 394 or 398 of alpha-amylase derived from B. amyloliquefaciens or a homologous position in a homologous alpha-amylase.

Modified Bacillus amyloliquefaciens alpha-amylases with altered thermal stability and activity have been disclosed (Lee S., et al., Comparison of the wild-type alpha-amylase and its variant enzymes in Bacillus amyloliquefaciens in activity and thermal stability, and insights into engineering the thermal stability of Bacillus alpha-amylase, J. Biochem. 139:1007-1015, published online 21. Jun. 2006) and (JP patent application no. 11-234813 published as 2000-135093).

### SUMMARY OF THE INVENTION

The present invention relates to alpha-amylolytic variants of a parent bacterial alpha-amylase (also denoted ‘parent alpha-amylase’), in particular variants exhibiting altered pH profile (relative to the parent), which is advantageous in connection with baking.

The inventors have found that the variants with altered pH profile improves the volume of the bread as compared to the parent bacterial alpha-amylase without causing unwanted side effects such as a gummy and inelastic crumb. Without being limited to any specific theory it is believed that the positive effect of the variants of the invention and the absence of unwanted side effects may be ascribed to a pH decrease in the dough during leavening, said decrease leading to at least a partial inactivation of the enzyme.

The invention further relates to DNA constructs encoding variants of the invention, to composition comprising variants of the invention, to methods for preparing variants of the invention, and to the use of variants and compositions of the invention, alone or in combination with other enzymes, in baking.

### DEFINITIONS

Alpha-amylase activity: The term “alpha-amylase activity” is defined herein as the endohydrolysis of 1,4-alpha-D-glucosidic linkages in polysaccharides containing three or more 1,4-alpha-linked D-glucose units.

Isolated polypeptide: The term “isolated polypeptide” as used herein refers to a polypeptide which is at least 20% pure, preferably at least 40% pure, more preferably at least 60% pure, even more preferably at least 80% pure, most preferably at least 90% pure, and even most preferably at least 95% pure, as determined by SDS-PAGE.

Identity: The relativity between two amino acid sequences or between two nucleotide sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between amino acids 1 to 512 of SEQ ID NO:2 (“reference sequence”) and a different amino acid sequence (“foreign sequence”) is calculated as the number of exact matches in an alignment of the two sequences, divided by the length of the “reference sequence” or the length of the “foreign sequence”, whichever is the shortest. The result is expressed in percent identity.

An exact match occurs when the “reference sequence” and the “foreign sequence” have identical amino acid residues in the same positions of the overlap (in the alignment example below this is represented by “|”). The length of a sequence is the number of amino acid residues in the sequence (e.g. the length of SEQ ID NO: 2 is 512).

In the alignment example below, the overlap is the amino acid sequence “HTWGER-NL” of Sequence 1; or the amino acid sequence “HGWGEDANL” of Sequence 2. In the example a gap is indicated by a “-”.

### Hypothetical Alignment Example:

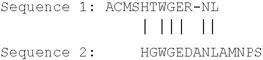

Coding sequence: When used herein the term “coding sequence” means a nucleotide sequence, which directly specifies the amino acid sequence of its protein product.

Expression: The term “expression” includes any step involved in the production of the polypeptide.

cDNA: The term “cDNA” is defined herein as a DNA molecule which lacks intron sequence. The cDNA can be prepared by reverse transcription from a mature, spliced, mRNA molecule obtained from a eukaryotic cell.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Expression vector: The term “expression vector” is defined herein as a linear or circular DNA molecule that comprises a polynucleotide encoding a polypeptide of the invention, and which is operably linked to additional nucleotides that provide for its expression.

Host cell: The term “host cell”, as used herein, includes any cell type which is susceptible to transformation, transfection, transduction, and the like with a nucleic acid construct comprising a polynucleotide of the present invention.

Mutation: The term “mutation” is defined herein as being a deletion, insertion or substitution of an amino acid in an amino acid sequence.

Nomenclature for variants: The nomenclature used for describing variants of the present invention is the same as the nomenclature used in WO 92/05249, i.e. the conventional one-letter codes for amino acid residues are used, and alpha-amylase variants of the invention are described by use of the following nomenclature:

Original amino acid(s): position(s): substituted amino acid(s)

According to this nomenclature, for instance the substitution of aspartic acid for asparagine in position 183 is shown as D183N, whereas a deletion of aspartic acid in the same position is shown as D183*.

### DETAILED DESCRIPTION OF THE INVENTION

### The Parent Bacterial Alpha-Amylase of the Invention

The parent bacterial alpha-amylase of the invention is an amino acid sequence having alpha-amylase activity and at least 80% identity with the B. amyloliquefaciens alpha-amylase having the amino acid sequence shown in SEQ ID NO: 2.

In a preferred embodiment the parent bacterial alpha-amylase of the invention has at least 85%, such as at least 90%, or at least 95% identity, more preferred at least 97%, such as at least 98% or even at least 99% with the amino acid sequences of SEQ ID NO:2. In a most preferred embodiment the parent alpha-amylase identical to the B. amyloliquefaciens alpha-amylase having the amino acid sequence shown in SEQ ID NO: 2.

For a parent alpha-amylase other than B. amyloliquefaciens alpha-amylase having the amino acid sequence shown in SEQ ID NO: 2, the corresponding positions in the parent alpha-amylase sequence of interest is identified by aligning to SEQ ID NO:2 using the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

In another preferred embodiment the parent bacterial alpha-amylase of the invention is encoded by a DNA sequence which hybridizes under at least medium stringency conditions, preferably medium-high stringency conditions, even more preferably high stringency conditions with a complementary strand of nucleotides 41 to 1069 or preferably nucleotides 1 to 1536 of SEQ ID NO:1 (J. Sambrook, E. F. Fritsch, and T. Maniatus, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides. Moreover, the subsequence may encode a polypeptide fragment which has alpha-amylase activity.

For long probes of at least 100 nucleotides in length, stringency conditions are defined as prehybridization and hybridization at 42° C. in 5×SSPE, 0.3% SDS, 200 ug/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

Under salt-containing hybridization conditions, the effective Tm is what controls the degree of identity required between the probe and the filter bound DNA for successful hybridization. The effective Tm may be determined using the formula below to determine the degree of identity required for two DNAs to hybridize under various stringency conditions.

Effective Tm=81.5+16.6(log M[Na+])+0.41(%G+C)−0.72(% formamide)

A 1% mismatch of two DNAs lowers the Tm by 1.4° C. To determine the degree of identity required for two DNAs to hybridize under medium stringency conditions at 42° C., the following formula is used:

% Homology=100−[(Effective Tm−Hybridization Temperature)/1.4]

### The Variant of the Invention

In a first aspect the variant of the invention has not more than 70% activity at pH 5 where the activity of the variant is defined to be 100% at pH 6.0, where the activity is measured after 15 minutes incubation at 37° C. using the Phadebas assay described in Example 3

Differential Scanning Calorimetri is applied to evaluate the thermostability of the bacterial alpha amylase variants of the invention as a lower Tm of a variant as compared to the parent bacterial alpha amylase corresponds to reduced thermostability of said variant. Therefore in a second aspect the variant of the invention has a melting point (Tm) of not more than 64° C. when measured by DSC in a buffer with 50 mM Na-acetate and 1 mM CaCl₂ at pH 5.5 as described in Example 5. In a preferred embodiment Tm is not more than 63° C., such as not more than 62° C. or not more than 61° C., and in a more preferred embodiment Tm is not more than 60° C., such as not more than 59° C. or not more than 58° C.; in a most preferred embodiment Tm is not more than 57° C., such as not more than 56° C. or 55° C.

In a further aspect the present invention relates to a variant of the parent bacterial alpha-amylase having alpha-amylase activity and comprising a mutation at the position corresponding to D183 when the amino acid sequence of the parent bacterial alpha-amylase is aligned with the amino acid sequence of SEQ ID NO:2. In a particular embodiment the mutation is the substitution D183N.

In a still further aspect the variant of the present invention may comprise at least one further substitution selected from the group consisting of P120, D204 and R249.

The total number of amino acid substitutions, deletions and/or insertions of amino acids of SEQ ID NO: 2 is 10, preferably 9, more preferably 8, more preferably 7, more preferably at most 6, more preferably at most 5, more preferably 4, even more preferably 3, most preferably 2, and even most preferably 1.

One or more of these substitutions may be conservative amino acid substitutions. A conservative amino acid substitution is within the context of the present application to be understood as an amino acid substitution that alters neither the pH-profile, nor the Tm as determined by DSC. In a preferred embodiment the conservative amino acid substitution is characterized in, that an amino acid is substituted with a different amino acid belonging to the same group of amino acids. The amino acids may for this purpose be divided in six groups: basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Conservative amino acid substitutions in variants of bacterial alpha-amylases of the present invention are also covered by the scope of the present invention.

### Polynucleotides

The present invention also relates to polynucleotides having nucleotide sequences which have a degree of identity to the mature polypeptide coding sequence of SEQ ID NO: 1 (i.e., nucleotides 41 to 1069) of at least 40, preferably at least 50, preferably at least 60%, preferably at least 65%, more preferably at least 70%, more preferably at least 75%, more preferably at least 80%, more preferably at least 85%, more preferably at least 90%, even more preferably at least 95%, and most preferably at least 97% identity, which encode an active polypeptide.

### Nucleic Acid Constructs

The present invention also relates to nucleic acid constructs comprising an isolated polynucleotide of the present invention operably linked to one or more control sequences which direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotides sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a polypeptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

### Expression Vectors

The present invention also relates to recombinant expression vectors comprising a polynucleotide of the present invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

### Host Cells

The present invention also relates to recombinant host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a unicellular microorganism, e.g., a prokaryote, or a non-unicellular microorganism, e.g., a eukaryote.

Examples of suitable host cells are the following: gram positive bacteria such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans or Streptomyces murinus; and gram-negative bacteria such as E. coli.

### Cloning a DNA Sequence Encoding an Alpha-Amylase

The DNA sequence encoding a parent alpha-amylase may be isolated from any cell or micro organism producing the alpha-amylase in question, using various methods well known in the art. First, a genomic DNA and/or cDNA library should be constructed using chromosomal DNA or messenger RNA from the organism that produces the alpha-amylase to be studied. Then, if the amino acid sequence of the alpha-amylase is known, homologous, labelled oligonucleotide probes may be synthesized and used to identify alpha-amylase-encoding clones from a genomic library prepared from the organism in question. Alternatively, a labelled oligonucleotide probe containing sequences homologous to a known alpha-amylase gene could be used as a probe to identify alpha-amylase-encoding clones, using hybridization and washing conditions of lower stringency.

Alternatively the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or R. K. Saiki et al. (1988).

### Methods of Production

The present invention also relates to methods for producing a polypeptide of the present invention, comprising cultivating a cell, harboring a polynucleotide encoding the polypeptide of the invention, under conditions conducive for production of the polypeptide; and recovering the polypeptide.

### Compositions

The present invention also relates to compositions comprising a polypeptide of the present invention. Preferably, the compositions are enriched in such a polypeptide. The term “enriched” indicates that the alpha-amylase activity of the composition has been increased, e.g., with an enrichment factor of 1.1.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

The additional enzymes of the composition may further be produced by micro organism belonging to the genus Bacillus, such as Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Baking

In a specific embodiment the variant of the present invention is used for baking.

### Dough

The dough of the invention generally comprises wheat meal or wheat flour and/or other types of meal, flour or starch such as corn flour, corn starch, rye meal, rye flour, oat flour, oat meal, soy flour, sorghum meal, sorghum flour, potato meal, potato flour or potato starch.

The dough of the invention may be fresh, frozen or par-baked.
The dough of the invention is normally leavened dough or dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae.

The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate.

The dough may comprise fat (triglyceride) such as granulated fat or shortening, but the invention is particularly applicable to a dough where less than 1% by weight of fat (triglyceride) is added, and particularly to a dough which is made without addition of fat.

The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin.

### Additional Enzymes

Optionally, an additional enzyme may be used together with the amylase. The additional enzyme may be an amylase, such as an a maltogenic amylase, amyloglucosidase, a beta-amylase, a cyclodextrin glucanotransferase, or the additional enzyme may be a peptidase, in particular an exopeptidase, a transglutaminase, a lipolytic enzyme, a cellulase, a hemicellulase, in particular a pentosanase such as xylanase, a protease, a protein disulfide isomerase, e.g., a protein disulfide isomerase as disclosed in WO 95/00636, a glycosyltransferase, a branching enzyme (1,4-alpha-glucan branching enzyme), a 4-alpha-glucanotransferase (dextrin glycosyltransferase) or an oxidoreductase, e.g., a peroxidase, a laccase, a glucose oxidase, a pyranose oxi-dase, a lipoxygenase, an L-amino acid oxidase or a carbohydrate oxidase.

The additional enzyme may be of any origin, including mammalian and plant, and preferably of microbial (bacterial, yeast or fungal) origin and may be obtained by techniques conventionally used in the art.
The maltogenic amylase may be derived from Bacillus stearothermiphilus as described in EP 494233 or a variant thereof as described in WO 99/43794.
The lipolytic enzyme may have lipase activity (EC 3.1.1.3), phospholipase A1 activity, phospholipase A2 activity and/or galactolipase activity.

### Baked Product

The process of the invention may be used for any kind of baked product pre-pared from dough, either of a soft or a crisp character, either of a white, light or dark type. Examples are bread (in particular white, whole-meal or rye bread), typically in the form of loaves or rolls, French baguette-type bread, pita bread, tortillas, cakes, pancakes, biscuits, cookies, pie crusts, crisp bread, steamed bread, pizza and the like.

### Baking Composition

The present invention further relates to a baking composition comprising flour together with the polypeptide of the invention. The baking composition may contain other dough-improving and/or bread-improving additives, e.g. any of the additives, including enzymes, mentioned above.

### Enzyme Preparation

The invention provides an enzyme preparation comprising a variant of a bacterial alpha-amylase, for use as a baking additive in the process of the invention. The enzyme preparation is preferably in the form of a granulate or agglomerated powder. It preferably has a narrow particle size distribution with more than 95% (by weight) of the particles in the range from 25 to 500 micro-m.

Granulates and agglomerated powders may be prepared by conventional methods, e.g. by spraying the amylase onto a carrier in a fluid-bed granulator. The carrier may consist of particulate cores having a suitable particle size. The carrier may be soluble or insoluble, e.g. a salt (such as sodium chloride or sodium sulfate), a sugar (such as sucrose or lactose), a sugar alcohol (such as sorbitol), starch, rice, corn grits, or soy.

### Materials

Chemicals used as buffers and substrates were commercial products of at least reagent grade.

### Enzymes:

### Amylases Tested:

Variant 1 is a bacterial alpha-amylase being identical to the Bacillus amyloliquefaciens alpha-amylase shown in SEQ ID NO: 2, except that the mutation D183N has been introduced.
Variant 2 is a bacterial alpha-amylase being identical to the Bacillus amyloliquefaciens alpha-amylase shown in SEQ ID NO: 2, except that the mutation D204N has been introduced.
Variant 3 is a bacterial alpha-amylase being identical to the Bacillus amyloliquefaciens alpha-amylase shown in SEQ ID NO: 2, except that the mutation D204S has been introduced.
Fungamyl (SEQ ID NO: 4) is a commercially available fungal alpha-amylase from Novozymes A/S.
BAN (SEQ ID NO: 2) is a commercially available bacterial alpha-amylase from Novozymes A/S.
All enzymes are at least 95% pure as determined by SDS-page.

### Media for Bacterial Growth

### Composition of BPX Medium:

| | | |
|---|---|---|
| Potato starch | 100 | g/l |
| Barley flour | 50 | g/l |
| BAN 5000 SKB | 0.1 | g/l |
| Sodium caseinate | 10 | g/l |
| Soy Bean Meal | 20 | g/l |
| Na₂HPO₄, 12 H₂O | 9 | g/l |
| Pluronic ™ | 0.1 | g/l |

### Substrates

Phadebas amylase test tablets, Art. No. 1302, Magle life science, Magle AB, Lund, Sweden.

### EXAMPLES

### Example 1

### Construction of Variants

B. amyloliquefaciens strains harbouring the relevant expression constructs are made by standard methods known in the art, such as, SOE-PCR using mutagenic primers to introduce site-specific amino-acid alterations, e.g., deletions, additions, or substitutions.

### Example 2

### Fermentation and Purification of Alpha-Amylase Variants

A B. amyloliquefaciens strain harbouring the relevant expression plasmid was streaked on an LB agar plate with 10 micro g/ml Chloramphenicol from −80° C. stock, and grown overnight at 37° C.

The colonies were transferred to 100 ml BPX media supplemented with 10 micro g/ml Chloramphenicol in a 500 ml shaking flask. The culture was shaken at 37° C. at 270 rpm for 4 days.

Cells and cell debris were removed from the fermentation broth by centrifugation at 4500 rpm in 20-25 minutes. Afterwards the supernatant was filtered to obtain a completely clear solution. The filtrate was concentrated and washed with water on an UF-filter (10000 cut off membrane) and at the end of the washing process the buffer was changed to 20 mM HEPES, 1 mM CaCl₂, pH 7. The final ion strength should be kept below 4 mS/cm. The UF-filtrate was applied on a sepharose F.F. and elution is carried out by linear gradient elution with NaCl in the same buffer. The fractions that contain the activity (measured by the Phadebas assay) are pooled, pH was adjusted to pH 7.5 and remaining color was removed by a treatment with 0.5% W/vol. active coal in 5 minutes.

### Example 3

### Assay for Alpha-Amylase Activity

Alpha-Amylase activity is determined by a method employing Phadebas® tablets as substrate. Phadebas tablets (Phadebas® Amylase Test, supplied by Magle life science, Lund, Sweden) contain a cross-linked insoluble blue-coloured starch polymer, which has been mixed with bovine serum albumin and a buffer substance and tableted.

For every single measurement one tablet was suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH or HCl). The test was performed in a water bath at the temperature of interest. The alpha-amylase to be tested was diluted in of 50 mM Britton-Robinson buffer in such a way that the absorbance upon incubation was in the range of 0.2 to 1.2 absorbance units. 1 ml of this alpha-amylase solution is added to the 5 ml 50 mM Britton-Robinson buffer. The starch is hydrolysed by the alpha-amylase giving soluble blue fragments. The absorbance of the resulting blue solution, measured spectrophotometrically at 620 nm, is a function of the alpha-amylase activity.

It is important that the measured 620 nm absorbance after 15 minutes of incubation (reaction time) is in the range of 0.2 to 1.2 absorbance units at 620 nm. In this absorbance range there is linearity between activity and absorbance (Lambert-Beer law). The dilution of the enzyme must therefore be adjusted to fit this criterion. Under a specified set of conditions (temperature, pH, reaction time, buffer conditions) 1 mg of a given alpha-amylase will hydrolyse a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

### Example 4

### Measuring the pH-Profile

The variant is tested at 37° C. at pH 4 to pH 10 in a Britton-Robinson buffer. Activity is determined according to the alpha-amylase activity assay described above. The following results were obtained (activity at pH 6 is set to 100%):

TABLE 1
pH profile for different alpha-amylases.

| | pH | | | | | | | | | | | | |
|---|---|---|---|---|---|---|---|---|---|---|---|---|---|
| A650 | 4 | 4.5 | 5 | 5.5 | 6 | 6.5 | 7 | 7.5 | 8 | 8.5 | 9 | 9.5 | 10 |
| BAN | 0 | 33 | 88 | 97 | 100 | 96 | 84 | 78 | 46 | 27 | 22 | 13 | 3.6 |
| Variant 1 | 0 | 20 | 67 | 89 | 100 | 94 | 89 | 67 | 35 | 24 | 15 | 8.9 | 1.9 |
| Variant 2 | 0.7 | 46 | 90 | 100 | 100 | 112 | 95 | 61 | 51 | 35 | 25 | 14 | 5.8 |
| Variant 3 | 0 | 25 | 87 | 93 | 100 | 92 | 81 | 65 | 44 | 30 | 22 | 13 | 5.8 |
| Fungamyl | 192 | 177 | 137 | 117 | 100 | 74 | 54 | 30 | 14 | 6.8 | 4.6 | 1.9 | 0 |

### Example 5

### Determination of Melting Point (Tm) by DSC

The thermostability in terms of melting point (Tm) of Fungamyl (SEQ ID NO: 4), BAN (SEQ ID NO: 2), Variant 1, Variant 2 and Variant 3 was determined by DSC. The buffer of the purified enzyme protein is changed to a buffer with 50 mM Na-acetate and 1 mM CaCl₂ at pH 5.5 using an Amicon ultra-15 centrifugal filter unit with an Ultracel 5 kDa membrane, art. No. UFC900524, Milipore, Ireland. The protein solution is the diluted to approximately 1 mg/mL.

The following results were obtained:

TABLE 2
Thermostability of different alpha-amylases.

| | Tm [° C.] |
|---|---|
| Fungamyl | 67 |
| Variant 1 | 64 |
| Variant 2 | 76 |
| Variant 3 | 72 |
| BAN (SEQ ID NO: 2) | 78 |

### Example 6

### Baking Trials

Bread was baked according to the straight dough method.

### Process Flow Straight Dough Procedure:

### Recipe (Example)

| Dough | % on flour basis |
|---|---|
| Ascorbic acid | to be optimized for each flour (50 ppm in this trial) |
| Yeast | 4 |
| Salt | 1.5 |
| Water | to be optimized for each flour (61% in this trial) |
| Wheat flour + | 100 (Pelikaan Menaba flour) |
| enzymes | |

### Procedure

1. Scaling of ingredients, addition of yeast, ascorbic acid and enzymes
2. Temperature adjustment, scaling and addition of water into mixer bowl
3. Addition of flour into mixer bowl
4. Mixing: 3 min at setting 1 and 7 min at setting 2 using a Diosna spiral mixer.
5. The dough is taken from the mixer bowl and the temperature is determined, the dough parameters are determined (dough evaluation after mixing) and the dough is molded on the molder.
6. The dough is given 20 min bench-time under plastic cover and the second dough evaluation is performed (dough parameters after bench-time)
7. The dough is scaled for roll maker plate (1500 g/30 rolls) and bread (350 g/bread) and molding there after.
8. The molded dough is given 15 minutes bench time covered in plastic
9. The dough for rolls are formed to a ˜34 cm round plate and put on a roll maker plate and rolls are formed in a rounder. The rolls are transferred to a silscone covered baking sheet.
   The dough for bread are shaped in a sheeter and transferred to pans which are put in baking sheet.
10. The bread and rolls are proofed at 32° C., 86% rh.
   The proofing time for rolls are 45 min
   The proofing time for bread is 55 min
11. The bread is baked at 230° C. with steam
   The rolls are baked for 22 min (damper opens after 12 min in order to let out the steam from the oven)
   The bread is baked for 35 min (damper opens after 25 min in order to let out the steam from the oven)
12. Bread for volume determination is baked in open pans while bread for texture measurements are baked in lidded pan for constant volume.
13. The bread is taken out of the pans after baking and put on a baking sheet.
14. The bread and rolls are allowed to cool down.
15. The bread and rolls are evaluated regarding volume, ascorbic acid factor, crust and crumb.

The enzymes tested, Variant 1, Variant 2 and Variant 3, were dosed at 0.05, 0.1, 0.2, 0.3 and 0.4 mg protein enzyme/kg. Bread with no enzyme was used as a control, and Fungamyl (1.5 mg protein enzyme/kg flour) was used as a benchmark.

The dough stickiness which is a sensory evaluation performed by an experienced baker where the control dough without enzyme is given the character 5 and the other doughs are judged compared to the control on a scale from 0 to 10 where 0 is little stickiness and 10 is very sticky.

The volume of rolls and bread was determined through rape seed displacement. The specific volume index was calculated according Equation 1:

Specific volume index=Specific volume of Bread with enzyme (in ml/g)/Specific volume of Bread without enzyme (in ml/g) Equation 1

The average specific volume of three controls dough was set to 100%. The specific volumes of the enzyme treated bread are average of double samples. The crumb elasticity was determined as described in U.S. Pat. No. 6,876,932.

### Results Obtained

The effect of the different amylases on roll and bread volume can be seen in Table 3 and Table 4. The effect on dough parameters can be found in Table 5.

The effect of the different amylases on bread crumb elasticity can be found in Table 6.

Variant 1 is able to give increase in specific volume index already at a dosage of 0.2 mg protein enzyme/kg flour, without the negative effects commonly seen for bacterial alpha amylases with excessive dough stickiness loss of crumb elasticity and gummy and sticky crumb.

The variants with higher activity at lower pH, Variant 2 and Variant 3, are also able to give increase in the specific volume index at low mg protein enzyme/kg flour but with the negative effects commonly seen for bacterial alpha amylases, namely low crumb elasticity and gummy and sticky crumb.

TABLE 3
Specific volume index [%] with enzyme treatment for rolls

| | | 15 FAU/kg | 0.05 | 0.1 | 0.2 | 0.3 | 0.4 |
|---|---|---|---|---|---|---|---|
| | No enzyme | (1.5 mg/kg) | mg/kg | mg/kg | mg/kg | mg/kg | mg/kg |
| No | 100 | | | | | | |
| enzyme | | | | | | | |
| Fungamyl | | 103 | | | | | |
| Variant 1 | | | 102 | 101 | 103 | 103 | 104 |
| Variant 2 | | | 102 | 104 | 105 | 105 | 109 |
| Variant 3 | | | 104 | 104 | 107 | 105 | 107 |

TABLE 4
Specific volume index [%] with enzyme treatment for bread

| | | 15 FAU/kg | 0.05 | 0.1 | 0.2 | 0.3 | 0.4 |
|---|---|---|---|---|---|---|---|
| | No enzyme | (1.5 mg/kg) | mg/kg | mg/kg | mg/kg | mg/kg | mg/kg |
| No | 100 | | | | | | |
| enzyme | | | | | | | |
| Fungamyl | | 104 | | | | | |
| Variant 1 | | | 101 | 103 | 106 | 105 | 106 |
| Variant 2 | | | 102 | 104 | 107 | 108 | 109 |
| Variant 3 | | | 104 | 104 | 105 | 106 | 107 |

TABLE 5
Dough stickiness after floor time for enzyme treated doughs.

| | | 15 FAU/kg | 0.05 | 0.1 | 0.2 | 0.3 | 0.4 |
|---|---|---|---|---|---|---|---|
| | No enzyme | (1.5 mg/kg) | mg/kg | mg/kg | mg/kg | mg/kg | mg/kg |
| No | 5 | | | | | | |
| enzyme | | | | | | | |
| Fungamyl | | 6 | | | | | |
| Variant 1 | | | 6 | 6 | 7 | 7 | 7 |
| Variant 2 | | | 6 | 6 | 7 | 7 | 8 |
| Variant 3 | | | 5 | 6 | 7 | 6 | 7 |

TABLE 6
Effect on elasticity measured texture analyser

| Day | 1 | 4 | 7 |
|---|---|---|---|
| Control | 62.2 | 54.9 | 52.7 |
| Fungamyl 15 [FAU/kg] | 60.9 | 55.0 | 53.6 |
| Variant 1 0.2 [mg/kg] | 60.3 | 54.4 | 52.5 |
| Variant 1 0.6 [mg/kg] | 58.8 | 53.1 | 51.5 |
| Variant 1 1 [mg/kg] | 57.9 | 52.6 | 50.5 |
| Variant 2 0.2 [mg/kg] | 53.8 | 50.7 | 49.5 |
| Variant 2 0.5 [mg/kg] | 49.1 | 49.0 | 49.5 |
| Variant 3 0.15 [mg/kg] | 52.6 | 50.3 | 50.1 |
| Variant 1 0.3 [mg/kg] | 48.6 | 49.6 | 49.7 |

### Sensory Evaluation

From the sensory evaluation similar results was seen as for the texture. Variant 1 did not give any sticky or gummy crumb at any of the tested dosages. The two other variants, Variant 2 and Variant 3, had clearly an effect on the elasticity of the bread crumb giving sticky and gummy crumb in the high dosages (0.5 and 0.3 mg/kg respectively). There was also a tendency towards gumminess and stickiness in the low dosage (0.2 and 0.15 mg/kg respectively).

### Example 7

### Activity of Multi-Substituted BAN Variants at pH 5 and 6

Variant 4 is a bacterial alpha-amylase being identical to the Bacillus amyloliquefaciens alpha-amylase shown in SEQ ID NO: 2, except that the mutations P120G, D183N have been introduced.

Variant 5 is a bacterial alpha-amylase being identical to the Bacillus amyloliquefaciens alpha-amylase shown in SEQ ID NO: 2, except that the mutations P120G, D204N have been introduced.

Variant 6 is a bacterial alpha-amylase being identical to the Bacillus amyloliquefaciens alpha-amylase shown in SEQ ID NO: 2, except that the mutations P120G, D204S have been introduced.

Variant 7 is a bacterial alpha-amylase being identical to the Bacillus amyloliquefaciens alpha-amylase shown in SEQ ID NO: 2, except that the mutations R249A, D183N have been introduced.

Variant 8 is a bacterial alpha-amylase being identical to the Bacillus amyloliquefaciens alpha-amylase shown in SEQ ID NO: 2, except that the mutations R249A, D204N have been introduced.

Variant 9 is a bacterial alpha-amylase being identical to the Bacillus amyloliquefaciens alpha-amylase shown in SEQ ID NO: 2, except that the mutations R249A, D204S have been introduced.

Variant 10 is a bacterial alpha-amylase being identical to the Bacillus amyloliquefaciens alpha-amylase shown in SEQ ID NO: 2, except that the mutations R249A, P120G, D204N have been introduced.

Variant 11 is a bacterial alpha-amylase being identical to the Bacillus amyloliquefaciens alpha-amylase shown in SEQ ID NO: 2, except that the mutations R249A, P120G, D204S have been introduced.

Variant 12 is a bacterial alpha-amylase being identical to the Bacillus amyloliquefaciens alpha-amylase shown in SEQ ID NO: 2, except that the mutations P120G, R249A have been introduced.

Variant 13 is a bacterial alpha-amylase being identical to the Bacillus amyloliquefaciens alpha-amylase shown in SEQ ID NO: 2, except that the mutations D183N, R437W have been introduced.

The activity of these different BAN variants 4-13 was measured at pH 5 and pH 6 using the phadebas method. The activity at pH 6 was set to 100% and the activity at pH5 was measured in relation to pH 6. For details of the assay see Example 3: Assay for Alpha-Amylase Activity.

The activity data at pH 5 and pH 6 can be found in the table below. Variant 4, 5, 6, 7, 12 and 13 are considerably de stabilized regarding pH. Variant 8 is just above the 70% limit claimed herein.

Variants 9 and 10, on the other hand, still retain a lot of activity at pH 5.

| | | Activity | Activity | Relative | Relative |
|---|---|---|---|---|---|
| Enzyme | | pH 5 | pH 6 | activity pH 5 | activity pH 6 |
| Variant | Mutation | [Abs] | [Abs] | [%] | [%] |
| 4 | P120G, D183N | 0.5785 | 1.1535 | 47.0 | 100 |
| 5 | P120G, D204N | 0.42 | 0.768 | 50.7 | 100 |
| 6 | P120G, D204S | 0.37 | 0.692 | 48.8 | 100 |
| 7 | R249A, D183N | 0.314 | 0.5655 | 50.3 | 100 |
| 8 | R249A, D204N | 0.5905 | 0.788 | 73.0 | 100 |
| 9 | R249A, D204S | 0.597 | 0.6925 | 84.7 | 100 |
| 10 | R249A, | 0.361 | 0.3725 | 88.5 | 100 |
| | P120G, D204N | | | | |
| 11 | R249A, | 0.318 | 0.4575 | 63.7 | 100 |
| | P120G, D204S | | | | |
| 12 | P120G, R249A | 0.4235 | 0.6215 | 59.9 | 100 |
| 13 | D183N, | 0.178 | 0.4355 | 29.8 | 100 |
| | R437W | | | | |

1. A variant of a parent bacterial alpha-amylase, wherein the parent bacterial alpha-amylase
a. has an amino acid sequence which has at least 80% identity with the amino acid sequence of the alpha-amylase of SEQ ID NO: 2, or
b. hybridizes under at least medium stringency conditions with a complementary strand of nucleotides 41 to 1069 of SEQ ID NO: 1;
and the variant has
c. alpha-amylase activity, and
d. not more than 70% activity at pH 5 when the activity of the variant is defined to be 100% at pH 6.0, where the activity is measured after 15 minutes incubation in 50 mM Britton-Robinson buffer at 37° C. using a Phadebas tablet assay.

2. The variant of claim 1 wherein the melting point, when measured by DSC in a buffer with 50 mM Na-acetate and 1 mM CaCl₂ at pH 5.5, is not more than 64° C.

3. The variant of claim 2 comprising a mutation at the position corresponding to D183.

4. The variant of claim 1 further comprising at least one mutation in a position selected from the group consisting of P120, D204 and R249.

5. An isolated polynucleotide comprising a nucleotide sequence which encodes the variant of claim 1.

6. A nucleic acid construct comprising the polynucleotide of claim 5 operably linked to one or more control sequences that direct the production of the variant in an expression host.

7. A recombinant expression vector comprising the nucleic acid construct of claim 6.

8. A recombinant host cell comprising the nucleic acid construct of claim 6.

9. A method for producing the variant of claim 1 comprising:
a. cultivating a cell, harbouring the polynucleotide of claim 5, under conditions conducive for production of the variant; and
b. recovering the variant.

10. The method of claim 9 wherein the cell is the recombinant host cell of claim 8.

11. (canceled)

12. A process for preparing dough or a baked product prepared from the dough, comprising incorporating into the dough the variant of claim 1.

13. A baking composition comprising the variant of claim 1 and a baking ingredient.

14. A composition comprising the variant of claim 1 and flour.

15. A composition comprising
a. the variant of claim 1, and
b. one or more components selected from the group consisting of leavening agent, milk powder, gluten, emulsifier, granulated fat and an oxidant.

16. The composition of claim 15 which is a dough.

17. The variant of claim 1 in a granulate form.
